(19) United States
(12) Patent Application Publication
(10) Pub. No.: US 2006/0004198 A1
(43) Pub. Date: Jan. 5, 2006

(54) Method for producing substituted phenylsulfonyl urea

(76) Inventors: Jan Vermehren, Idstein (DE); Mark James Ford, Bad Soden (DE); Gunter Schlegel, Leverkusen (DE); Iswald Ort, Metzer, Leverkusen (DE); Boerries Kuebel, Kelkheim (DE)

Correspondence Address: William F Lawrence;Frommer Lawrence & Haug, 745 Fifth Avenue, New York, NY, 10151, US

(21) Appl. No.: 10/511,968

(22) PCT Filed: Apr. 16, 2003
(86) PCT No.: PCT/EP03/03980

§ 371 Date: Aug. 26, 2005

(30) Foreign Application Priority Data
Apr. 26, 2002 (DE) 102 18 704.5

(51) Int. Cl. C07D 43/02

(52) U.S. Cl. 544/209; 544/329

(57) ABSTRACT

The invention relates to a preparation process for the compound (I) or a salt thereof 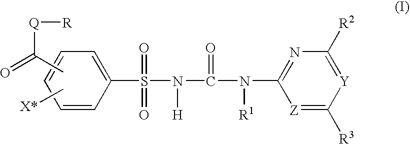
where Q, X*, Y, Z, R, R¹, R² and R³ are as defined in claim 1, 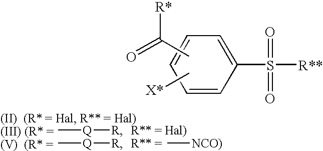 which comprises a) monoesterifying a compound (II) where Hal=a halogen atom, giving (III), b) either b1) ammonolysing and phosgenating this compound to give (V) or b2) converting this compound with cyanate into (V) and
c) converting the resulting compound (V) with the aminoheterocycle H₂N-Het (Het=heterocycle as in (I)) into the compound (I) or a salt thereof. Some compounds (II) and some intermediates (VIII), stabilized with N-heteroaromatic compounds, of the compounds (V) are novel. The compounds (II) can be obtained from the corresponding sulfobenzoic acids by reaction with acid halides of sulfur or phosphorus in nonpolar organic solvents.

The invention relates to the technical field of chemical processes for preparing compounds from the group of the herbicidal phenylsulfonylureas, and intermediates thereof.

A number of substituted phenylsulfonylureas have been described as herbicides and plant growth regulators. From the group of the phenylsulfonylureas, those having a carboxyl group or a carboxylic acid derivative group on the phenyl ring are synthetically particularly demanding. Of interest are the compounds, known from EP-A-007687 or WO-A-92/13845, of the formula (I) and salts thereof 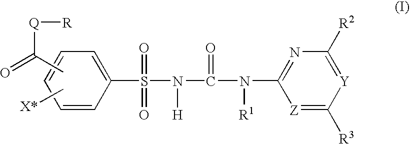
where
Q is oxygen or sulfur,
X* is hydrogen, halogen, cyano, nitro, (C₁-C₃)-alkyl or methoxy, preferably hydrogen or iodine, in particular iodine,
Y,Z independently of one another are CH or N, where Y and Z are not simultaneously CH,
R is hydrogen, (C₁-C₁₂)-alkyl, (C₂-C₁₀)-alkenyl, (C₂-C₁₀)-alkynyl, (C₁-C₆)-alkyl which is mono- to tetra-substituted by radicals selected from the group consisting of halogen, (C₁-C₄)-alkoxy, (C₁-C₄)-alkylthio, CN, [(C₁-C₄)-alkoxy]carbonyl and (C₂-C₆)-alkenyl, or (C₃-C₈)-cycloalkyl which is unsubstituted or substituted by radicals selected from the group consisting of (C₁-C₄)-alkyl, (C₁-C₄)-alkoxy, (C₁-C₄)-alkylthio and halogen, (C₅-C₈)-cycloalkenyl, phenyl-(C₁-C₄)-alkyl which is unsubstituted in the phenyl radical or substituted by one or more radicals selected from the group consisting of halogen, (C₁-C₄)-alkyl, (C₁-C₄)-alkoxy, (C₁-C₄)-haloalkyl, (C₁-C₄)-alkylthio [(C₁-C₄)-alkoxy]carbonyl, [(C₁-C₄)-alkyl]carbonyloxy, carbamoyl, [(C₁-C₄)-alkyl]carbonylamino, [(C₁-C₄)-alkyl]aminocarbonyl, di-[(C₁-C₄)-alkyl]aminocarbonyl and nitro, or a radical of the formulae A-1 to A-10 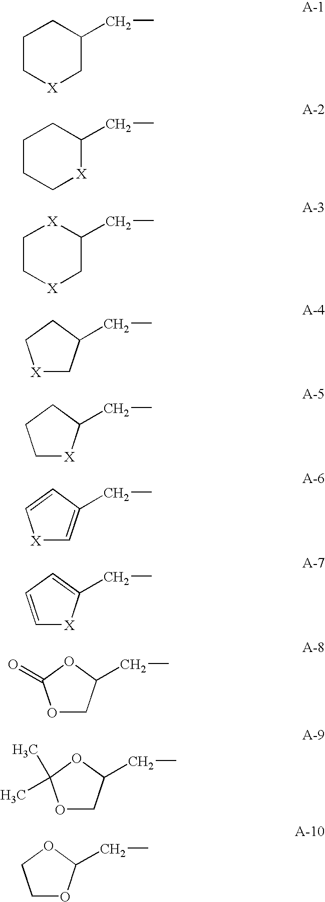
   where in the formulae A-1 to A-10 the radical X or the radicals X independently of one another is/are O, S, S(O) or SO₂,
R¹ is hydrogen or (C₁-C₃)-alkyl,
R² is hydrogen, halogen, (C₁-C₃)-alkyl or (C₁-C₃)-alkoxy, where each of the two last-mentioned radicals is unsubstituted or mono- or polysubstituted by halogen or (C₁-C₃)-alkoxy,
R³ is hydrogen, halogen, (C₁-C₃)-alkyl, (C₁-C₃)-alkoxy or (C₁-C₃)-alkylthio, where each of the three last-mentioned radicals is unsubstituted or mono- or polysubstituted by halogen or mono- or disubstituted by (C₁-C₃)-alkoxy or (C₁-C₃)-alkylthio, or a radical of the formula NR⁴R⁵, (C₃-C₆)-cycloalkyl, (C₂-C₄)-alkenyl, (C₂-C₄)-alkynyl, (C₃-C₄)-alkenyloxy or (C₃-C₄)-alkynyloxy,
R⁴ and R⁵ independently of one another are hydrogen, (C₁-C₄)-alkyl, (C₃-C₄)-alkenyl, (C₁-C₄)-haloalkyl or (C₁-C₄)-alkoxy.

The salts of the compounds (I) are preferably compounds in which the hydrogen atom in the SO₂NH group of the sulfonylurea is replaced by a cation, preferably a physiologically acceptable cation which can be used in crop protection, in particular an alkali metal or alkaline earth metal cation or an unsubstituted or substituted ammonium ion, including quaternary ammonium ions. Examples of cations are the sodium ion, the potassium ion and the ammonium ion.

Salts of the compounds of the formula (I) can be formed by adding a suitable inorganic or organic acid, such as, for example, HCl, HBr, H₂SO₄ or HNO₃, but also oxalic acid or a sulfonic acid, to a basic group, such as, for example, amino or alkylamino. Suitable substituents, which are present in deprotonated form, such as, for example, sulfonic acids or carboxylic acids, are capable of forming inner salts with groups which for their part can be protonated, such as amino groups.

Salts can also be formed by replacing the hydrogen of a suitable functional group, such as, for example, the carboxyl group, by a cation suitable for agriculture. These salts are, for example, metal salts, in particular alkali metal salts or alkaline earth metal salts, in particular sodium salts and potassium salts, or else ammonium salts, salts with organic amines or quaternary ammonium salts.

Of particular interest are compounds of the formula (I) or salts thereof and their preparation, where the group of the formula —CO-Q-R is located in the position ortho to the sulfonyl group of the sulfonylurea (1). Preference is given to compounds (I) or salts thereof in which Q=oxygen, X*=hydrogen or halogen, preferably iodine, R=(C₁-C₄)-alkyl, (C₂-C₄)-alkenyl, (C₂-C₄)-alkynyl, (C₁-C₄)-haloalkyl, or (C₁-C₄)-alkoxy-(C₁-C₄)-alkyl, preferably methyl or ethyl, in particular methyl. Preference is furthermore given to compounds (I) and salts thereof in which the group of the formula —CO-Q-R is located in the position ortho to the sulfonyl group of the sulfonylurea, X*=halogen, preferably iodine, and X* is located in the position para to the group of formula —CO-Q-R. Here and generally, preference is furthermore given to compounds (I) and salts thereof in which Z is a nitrogen atom and Y is a nitrogen atom or a group of the formula CH, and Y is preferably a nitrogen atom.

From WO-A-92/13845 and WO 01/23368 and literature cited therein, it is known that phenylsulfonyl halides substituted by carboxylic acid derivative radicals on the phenyl ring and additionally by other radicals such as halogen can be converted by ammonolysis into sulfonamides which, after phosgenation to isocyanates and subsequent reactions, are converted with heterocyclic amino compounds into sulfonylureas of the formula (I) or salts thereof.

The starting material (the phenylsulfonyl chloride in question) can, according to WO 92/13845, be prepared from substituted aminobenzoic acids by esterification, diazotization, reaction with SO₂ in the presence of Cu catalysts (Meerwein reaction) and oxidative cleavage of the resulting disulfide with gaseous chlorine in hydrochloric acid.

However, the known reaction path is unsatisfactory with respect to the yield and the number of steps. Providing the multifunctional phenylsulfonyl chloride, in particular, is complicated and not optimal in terms of yield. Moreover, the subsequent phosgenation requires particular complicated techniques for its practice and for process control. Accordingly, it is an object of the present invention to provide an alternative process which, compared to the known processes, can be carried out advantageously with respect to one aspect, preferably with respect to a plurality of aspects.

The present invention provides a process for preparing the phenylsulfonylureas of the formula (I) mentioned and salts thereof, which comprises
a) converting a compound of the formula (II) 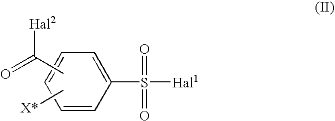
   where
   Hal¹ is a halogen atom, preferably chlorine or bromine, in particular chlorine,
   Hal² is a halogen atom, preferably chlorine or bromine, in particular chlorine, and
   X* is as defined in formula (I)
   by reaction with a compound of the formula R-Q-H or a salt thereof into a compound of the formula (III) 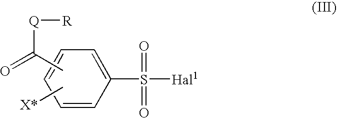
      where R, Q and X are as defined in formula (I) and Hal¹ is as defined in formula (II), and
   (b) with or without intermediate isolation either
      (b1) ammonolysing the resulting compound (III) to give the sulfonamide of the formula (IV) 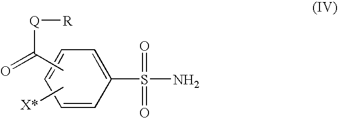
      where R, Q and X* are as defined in formula (III),
      and converting the compound (IV) with or without intermediate isolation with phosgene into the phenylsulfonyl isocyanate of the formula (V) 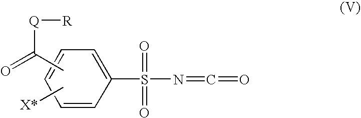
      where R, Q and X* are as defined in formula (III), or
      (b2) converting the resulting compound (III) with a cyanate, for example an alkali metal cyanate, into the isocyanate of the formula (V) or a solvated (stabilized) derivative thereof, and
   (c) converting the isocyanate of the formula (V) or its stabilized derivative, with or preferably without intermediate isolation, with a heterocyclic amine of the formula (VI) 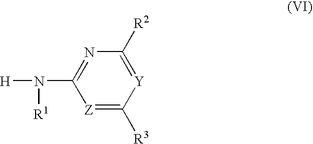
      where R¹, R², R³, Y and Z are as defined in formula (I),
      into the sulfonylurea of the formula (I) or a salt thereof.

Some compounds of the formula (II) (certain dihalides) are novel and also form part of the subject matter of the invention. The preparation of the compounds of the formula (II) (dihalides) also forms part of the subject matter of the invention (see below:).

The invention also provides the process according to sub-step (a) (selective esterification) using the compounds (II).

The invention also provides the process according to sub-step (b2) (conversion using cyanate), preferably in combination with sub-step (c).

In the definitions of the formulae (I) to (VI) and all formulae below, the radicals alkyl, alkoxy, haloalkyl, haloalkoxy, alkylamino and alkylthio and the corresponding unsaturated and/or substituted radicals can in each case be straight-chain or branched in the carbon skeleton.

Unless specified otherwise, the lower carbon skeletons, for example those having 1 to 6 carbon atoms, in particular 1 to 4 carbon atoms, or in the case of unsaturated groups those having 2 to 6 carbon atoms, in particular 2 to 4 carbon atoms, are preferred in these radicals. Alkyl radicals, including the composite meanings such as alkoxy, haloalkyl etc., denote, for example, methyl, ethyl, n- or i-propyl, n-, i-, t- or 2-butyl, pentyls, hexyls, such as n-hexyl, i-hexyl and 1,3-dimethylbutyl, heptyls, such as n-heptyl, 1-methylhexyl and 1,4-dimethylpentyl; alkenyl and alkynyl radicals have the meaning of the possible unsaturated radicals which correspond to the alkyl radicals and which contain at least one double bond or triple bond, preferably one double bond or triple bond. Alkenyl denotes, for example, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl; alkynyl denotes, for example, propargyl, but-2-yn-1-yl, but-3-yn-1-yl, 1-methylbut-3-yn-1-yl.

Cycloalkyl denotes a carbocyclic saturated ring system having preferably 3 to 8 carbon atoms, with preference 3 to 6 carbon atoms, for example cyclopropyl, cyclobutyl, cyclopentyl or cyclohexyl.

Halogen denotes, for example, fluorine, chlorine, bromine or iodine. In the definitions of radicals, “halogen” denotes a halogen radical, i.e. a halogen atom. Haloalkyl, -alkenyl and-alkynyl denote alkyl, alkenyl and alkynyl, respectively, which are partially or fully substituted by halogen, preferably by fluorine, chlorine and/or bromine, in particular fluorine or chlorine (by identical or different halogen atoms), for example monohaloalkyl (=monohalogenalkyl), perhaloalkyl, CF₃, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; this applies correspondingly to haloalkenyl and other halogen-substituted radicals.

Aryl is a carbocyclic aromatic system, for example a mono-, bi- or polycyclic aromatic system, for example phenyl, naphthyl, tetrahydronaphthyl, indenyl, indanyl, pentalenyl, fluorenyl and the like, preferably phenyl.

A hydrocarbon radical contains exclusively carbon atoms and hydrogen atoms and may be straight-chain, branched or cyclic, saturated, unsaturated or aromatic or may contain a combination of identical or different radicals of the hydrocarbon radicals mentioned above. “Hydrocarbon radical” embraces, for example, the radicals alkyl, alkenyl, alkynyl, cycloalkyl, cycloalkenyl, cycloalkylalkyl, cycloalkenylalkyl, aryl, such as phenyl or naphthyl, benzyl, phenethyl, etc. A hydrocarbon radical contains preferably 1 to 30 carbon atoms, in particular 1 to 24 carbon atoms, unless defined otherwise.

If a skeleton is substituted “by one or more radicals” from a list of radicals (=group) or a generically defined group of radicals, this includes in each case the simultaneous substitution by a plurality of identical and/or structurally different radicals from the group or the generically defined group.

Substituted radicals, such as a substituted hydrocarbon radical, for example a substituted alkyl, alkenyl, alkynyl, aryl, phenyl or benzyl radical, denote, for example, substituted radicals which are derived from the unsubstituted skeleton, the substituents being, for example, one or more, preferably 1, 2 or 3, radicals from the group consisting of halogen, alkoxy, alkylthio, hydroxyl, amino, nitro, carboxyl, cyano, azido, alkoxycarbonyl, alkylcarbonyl, formyl, carbamoyl, mono- and dialkylaminocarbonyl, substituted amino, such as acylamino, mono- and dialkylamino, alkylsulfinyl and alkylsulfonyl and, in the case of cyclic radicals, also alkyl, haloalkyl, alkylthioalkyl, alkoxyalkyl, unsubstituted or substituted mono- and dialkylaminoalkyl and hydroxyalkyl.

Preference is given to substituents selected from the group consisting of halogen, alkoxy, alkylthio, hydroxyl, amino, nitro, cyano, mono- and dialkylamino and, in the case of cyclic radicals, also alkyl and haloalkyl;

the term “substituted radicals” such as substituted hydrocarbon radicals, such as substituted alkyl, etc., includes as substituents in addition to the saturated hydrocarbon-containing radicals mentioned the corresponding unsaturated aliphatic and aromatic radicals, such as unsubstituted or substituted alkenyl, alkynyl, alkenyloxy, alkynyloxy, phenyl, phenoxy, etc. In the case of substituted cyclic radicals having aliphatic moieties in the ring, this also includes cyclic systems having substituents which are attached to the ring via a double bond, for example those which are substituted by an alkylidene group such as methylidene or ethylidene. The substituents mentioned by way of example (“first substituent level”) can, if they contain hydrocarbon-containing moieties, be, if appropriate, substituted further in these moieties (“second substituent level”), for example by one of the substituents defined for the first substituent level. Corresponding further substituent levels are possible. The term “substituted radical” preferably embraces only one or two substituent levels.

In the substituents mentioned, preference is in each case given to the number of carbon atoms mentioned above as being preferred for radicals having hydrocarbon moieties.

Some of the compounds of the formula (II) are known. Thus, U.S. Pat. No. 4,110,373 describes the reaction of unsubstituted or substituted benzotrichlorides with oleum. Mentioned in this publication is, inter alia, the preparation of 4-chloro-3-chlorosulfonylbenzoyl chloride and 3-chloro-5-chlorosulfonylbenzoyl chloride.

Furthermore, NL-A-7603612 discloses the preparation of 2-chlorosulfonylbenzoyl chloride from 2-sulfobenzoic acid by reaction with phosgene as halogenating agent in polar aprotic solvents such as DMF. Considerable amounts of dichlorotolylsultone (3,3-dichloro-1,1-dioxobenzo-1-thia-2-oxolane) are formed as byproduct. The process is generally also described for derivatives of sulfobenzoic acids which are additionally halogenated or nitrated at the benzene ring.

The compounds (I) and (III) to (VI) are described in principle and in some cases specifically in the publications EP-A-007687, WO-A-92/13845 and WO-A-01/23368 and in the literature cited therein. With respect to the process procedures and the preferred compounds of the formula (I) and their precursors mentioned therein, the content of WO-A-92/13845 and WO-A-01/23368 is particularly included by reference into the present description and invention.

Particularly preferred for the preparation process are compounds of the formula (II) in which Q=oxygen, X*=hydrogen or halogen, preferably iodine, R=(C₁-C₄)-alkyl, (C₂-C₄)-alkenyl, (C₂-C₄)-alkynyl, (C₁-C₄)-haloalkyl, or (C₁-C₄)-alkoxy(C₁-C₄)-alkyl, preferably methyl or ethyl, in particular methyl. Preferably Hal¹=a chlorine atom and Hal²=a chlorine atom.

Preference is also given to preparation processes with compounds of the formula (II) in which the carbonyl halide group is in the position ortho to the sulfonyl halide group. Preference is furthermore given to preparation processes with compounds of the formula (II) in which the carbonyl halide group is in the position ortho to the sulfonyl halide group and X*=a halogen atom, preferably iodine, in the position para to the carbonyl halide group.

Preference is therefore also given to processes in which compounds of the formula (IIa) are used in step a) as compounds of the formula (II): 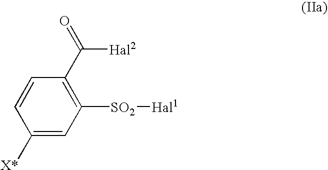
where Hal¹ is a halogen atom, preferably a chlorine or bromine atom, in particular a chlorine atom, Hal² is a halogen atom, preferably a chlorine or bromine atom, in particular a chlorine atom, and X* is as defined in formula (I), preferably halogen, in particular iodine.

Preference is generally given to processes with intermediates and compounds of the formula (I) whose substitution pattern on the phenyl ring corresponds to that of compounds (IIa).

Particular preference is also given to processes according to the invention which have a combination of features mentioned as being preferred.

The reaction according to the invention of the compound of the formula (II) with a compound of the formula R-Q-H or a salt thereof to give the compound of the formula (III) is a selective reaction of the dihalide with the nucleophile R-Q-H.

The reaction is carried out using an alcohol or thioalcohol (Q=oxygen or sulfur) and/or a salt thereof, where the salt is employed, for example, directly, or can be generated in the reaction mixture from the alcohol in the presence of another base. The reaction conditions are expediently chosen such that side reactions at the sulfonyl halide group are, as far as possible, avoided. Possible side reactions are, for example, the esterification of the sulfonyl halide giving the sulfonic acid ester, with subsequent intermolecular transesterification with formation of a sulfonic acid group and further subsequent reactions.

Relatively good yields of the desired monoester (III) of the substituted halosulfonylbenzoic acid are obtained, for example, when the reaction is carried out in an inert organic solvent and/or diluent (hereinbelow referred to in short as “solvent”), with temperature control. Suitable inert organic solvents are, for example, relatively unpolar aprotic organic solvents, such as
aliphatic and aromatic hydrocarbons, such as, for example, mineral oils, petroleum ether, n-pentane, n-hexane, cyclopentane, cyclohexane or toluene, xylenes, mesitylene, naphthalene derivatives, Solvesso® 200 (high-boiling mixture of aromatic compounds);
halogenated aliphatic and aromatic hydrocarbons, such as methylene chloride, dichloroethane or chlorobenzene, chlorotoluene or dichlorobenzene or
mixtures of the solvents mentioned.

For larger batches, the higher-boiling organic solvents such as toluene, xylene, mesitylene, chlorobenzene, chlorotoluene or dichlorobenzene or mixtures thereof are generally employed.

The alcohols or thioalcohols used are the compounds R-Q-H which correspond to the radical R-Q in formula (I). Preference is given here to the (C₁-C₄)-alkanols, such as methanol, ethanol, n-propanol, isopropanol, n-butanol, isobutanol, in particular methanol and ethanol. In general, 1 molar equivalent or an excess, for example 1 to 20 molar equivalents, preferably 1 to 8 molar equivalents, in particular 1 to 6 molar equivalents, of the compound R-Q-H are employed per mole of the compound of the formula (II) (dihalide, for example dichloride). In the case of methanol, preference is given to using 1 to 8 molar equivalents, in particular 1 to 6 molar equivalents, very particularly 3 to 6 molar equivalents.

Suitable salts of the alcohols or thioalcohols are, for example, the alkali metal salts, preferably the sodium or potassium salts, or the alkaline earth metal salts. In general, 1 molar equivalent or an excess, for example 1 to 10 molar equivalents, preferably 1 to 3 molar equivalents, in particular 1 to 2 molar equivalents, of the salt of the compound R-Q-H are employed per mole of the compound of the formula (II) (dihalide). In the case of a salt of methanol, for example sodium methoxide, preference is given to using 1 to 10 molar equivalents, in particular 1 to 3 molar equivalents, very particularly 1 to 2 molar equivalents, of methoxide.

Depending on the components and their proportion in the reaction mixture, the reaction temperature for the esterification can expediently be optimized by preliminary experiments and is usually in the range of from −20° C. to 100° C. If an alcohol or thioalcohol, preferably a (C₁-C₄)-alkanol, in particular methanol, is used for the esterification, a suitable reaction temperature is generally in the range of from −10° C. to 70° C., preferably from 20 to 40° C. If salts of the compounds R-Q-H are used, the optimum reaction temperature is in most cases comparably lower. A suitable reaction temperature for the esterification with a salt, preferably an alkali metal salt or alkaline earth metal salt of a (C₁-C₄)-alkanol or else thioalcohol, in particular sodium methoxide or potassium methoxide, is generally in the range of from −20° C. to 50° C., preferably from −10 to 35° C., in particular from 0 to 25° C.

The reaction mixture can be worked up by customary methods. After the reaction with an excess of alcohol has ended, excess alcohol can be distilled off, for example, under reduced pressure, and the reaction mixture can then be poured into water to remove the salt formed, and the product can then be extracted with an organic solvent. Alternatively, the mixture can be added directly to water and be extracted with a solvent.

The further reaction of the resulting halosulfonylbenzoic acid ester (III) (for example chlorosulfonylbenzoic acid ester) can be carried out according to or analogously to the known procedures as described, for example, in WO-A-92/13845 and WO 01/23368 and the literature cited therein. The known procedure involves the ammonolysis of the compound of the formula (III) to give the sulfonamide of the formula (IV), the phosgenation of the compound (IV) to give the phenylsulfonyl isocyanate of the formula (V) and a subsequent addition reaction (coupling) with a heterocyclic amine of the formula (VI) to give the sulfonylurea of the formula (I) or its salt. For the procedures for carrying out the ammonolysis, phosgenation and coupling, specific reference is made to WO 01/23368.

As an alternative to the above procedure, the compound of the formula (III) can be reacted with a cyanate, for example a cyanate metal salt, in particular an alkali metal cyanate, to give the isocyanate of the formula (V) or a solvated (stabilized) derivative thereof.

Suitable cyanates are cyanates having cations from the group of the metal cations or the organic cations, such as sterically hindered organic ammonium ions. Preference is given, for example, to alkali metal cyanates, preferably sodium cyanate and potassium cyanate, or else alkaline earth metal cyanates. The cyanate or cyanate mixture used is expediently employed in an amount which is sufficient for the conversion into the compound (V). In general, an equimolar amount of cyanate or a slight excess, preferably from 1 to 2 molar equivalents, in particular from 1 to 1.5 molar equivalents, of cyanate, based on the compound (III), is sufficient for this purpose.

The reaction with the cyanate is generally carried out in an aprotic polar solvent. Suitable solvents and/or diluents are aprotic organic solvents which are inert under the reaction conditions, for example
ethers, such as diethyl ether, di-n-propyl ether, diisopropyl ether, methyl-tert-butyl ether, tetrahydrofuran (THF), dioxane, alkylene glycol monoalkyl ethers and alkylene glycol dialkyl ethers, such as, for example, propylene glycol monomethyl ether, propylene glycol monoethyl ether, ethylene glycol monomethyl ether or ethylene glycol monoethyl ether, dimethoxyethane, diglyme, triglyme and tetraglyme;
amides, such as dimethylformamide (DMF), dimethylacetamide and N-methylpyrrolidone;
ketones, such as acetone, cyclohexanone, methyl isobutyl ketone (MIBK);
nitriles, such as acetonitrile, propionitrile, butyronitrile and benzonitrile;
sulfoxides and sulfones, such as dimethyl sulfoxide (DMSO) and sulfolane and mixtures of two or more of the abovementioned solvents or diluents.

Of particular interest with regard to industrial implementation are generally those solvents which are readily removable from the product by distillation.

Depending on the particle size of the cyanate, provided it is present in the reaction mixture as a solid, the reaction time may vary.

Preference is given to aprotic solvents selected from the group of the ethers, for example diisopropyl ether and methyl tert-butyl ether, ketones, for example methyl isobutyl ketone (MIBK) and nitrites, such as acetonitrile.

In most cases, it is advantageous to carry out the reaction to give the isocyanate of the formula (V) with addition of N-heteroaromatic compounds (nitrogen heterocycles) as catalyst or stabilizer in the reaction mixture, for example using pyridine and pyridine derivatives of the formula (VII) 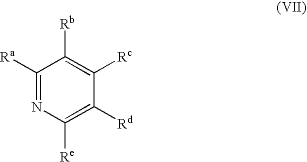
where
R^{a}, R^{b}, R^{c}, R^{d} and R^{e} each independently of one another are hydrogen, (C₁-C₆)-alkyl, (C₂-C₆)-alkenyl, (C₂-C₆)-alkynyl or (C₁-C₆)-alkoxy or two adjacent radicals together with the linking carbon atoms of the first ring form a fused-on carbocyclic ring having 4 to 8 carbon atoms or a heterocyclic ring having 4 to 8 ring atoms and 1, 2 or 3 heteroring atoms selected from the group consisting of N, O and S. Preferably, R^{a}, R^{b}, R^{c}, R^{d} and R^{e} each independently of one another are hydrogen, or one, two or three of the radicals are (C₁-C₄)-alkyl or (C₁-C₄)-alkoxy, in particular methyl or ethyl, and the other radicals are each hydrogen.

Examples of suitable N-heteroaromatic compounds are pyridine and substituted pyridines, such as alkylpyridines, for example picolines (e.g. 2-methylpyridine, 3-methylpyridine or 4-methylpyridine) or lutidines (e.g. 2,4-dimethylpyridine, 2,6-dimethylpyridine, 2,3-dimethylpyridine, 2,5-dimethylpyridine, 3,4-dimethylpyridine or 3,5-dimethylpyridine), or mixtures thereof. The amount of N-heteroaromatic compounds may vary within a wide range. Expediently, from 0.8 to 2 molar equivalents, preferably from 0.9 to 1.5 molar equivalents, in particular from 1 to 1.3 molar equivalents, of the N-heteroaromatic compound are employed per mole of the halosulfonylbenzoic acid ester (III), preferably chlorosulfonylbenzoic acid ester, based on one mole of the isocyanate (V) to be generated.

In the case of Y=CH (pyrimidin-2-yl), it may be sufficient to add catalytic amounts of pyridine or a pyridine derivative when preparing the isocyanate in the presence of a cyanate.

If a stabilizer from the group of the N-heteroaromatic compounds is added, all or some of the product is not present in the form of the isocyanate but in the form of an intermediate stable in solution which, if a pyridine (derivative) of the formula (VII) is used which has been mentioned as being preferred, is a compound of the formula (VIII) 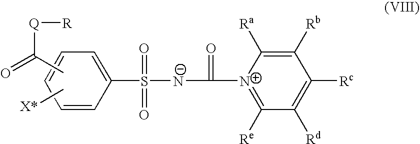
where R, R^{a}, R^{b}, R^{c}, R^{d}, R^{e}, Q and X* are as defined in formula (III) or formula (VII). Correspondingly, if the stabilizer used is pyridine, the intermediate of the formula (VIIIa) 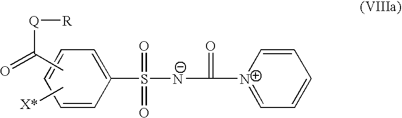
is formed where
R, Q and X* are as defined in formula (III).

The intermediates of the formulae (VIII) and (VIIIa) are novel and therefore also form part of the subject matter of the invention. Particular preference is given to the intermediates of the formula (VIIIb) 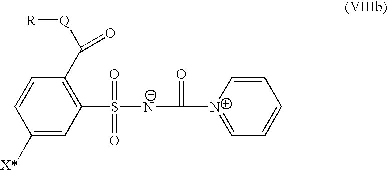
where
R, Q and X* are as defined in formula (III).

The compounds of the formulae (VIII), (VIIIa) and (VIIIb) can be detected, for example, spectroscopically. They are distinguished by a characteristic shift of the band of the carbonyl vibration in the infrared spectrum, compared to the carbonyl band of the corresponding isocyanates of the formula (V).

The reaction temperature for the reaction of the compound (III) with the cyanate can be varied within wide limits and can be optimized in preliminary experiments. Moderate values in the range of from −30° C. to 70° C., in particular from −10° C. to 30° C., are preferred.

Analogously to the isocyanate from variant (b1), the isocyanate (V) can then be reacted with a heterocyclic amine (VI) to give the sulfonylurea (I). If a catalyst or stabilizer is added, it is expedient to initially neutralize using an acid, preferably an anhydrous acid, for example hydrogen chloride or an organic acid.

In one variant of the process, the reaction mixture from the preparation of the isocyanate by the cyanate process is used directly for coupling the isocyanate or stabilized isocyanate to give the sulfonylurea of the formula (I). To this end, any N-heteroaromatic compounds present are neutralized by addition of an acid (as mentioned above), and the heterocyclic amine (VI) is added to the reaction mixture. Alternatively, the heterocyclic amine (VI) can be added, followed by addition of the acid to neutralize the catalyst/stabilizer.

The reactions of the compounds (V) and (VI) are generally carried out in an organic solvent. Solvents suitable for this purpose are polar or relatively unpolar aprotic solvents. Preference is given to using the same solvents for the reaction as for the preparation of the isocyanate (V).

The reaction is preferably carried out at a temperature in the range of from 0° C. to the boiling point of the solvent, preferably from 0° C. to 100° C., in particular from 20 to 80° C., very particularly from 20 to 40° C.

Based on one mole of sulfonyl isocyanate of the formula (I), preference is given to using from 1 to 1.2 mol, in particular from 1 to 1.1 mol, very particularly from 1 to 1.05 mol, of amine of the formula (VI).

Work-up of the reaction mixture from the coupling can be carried out by customary methods, the sulfonylureas of the formula (I) being able to be isolated, for example, as non-salts or—after reaction with bases or, if appropriate, also acids—as salts.

Using the process according to the invention, it is possible to prepare sulfonylureas (I) or salts thereof by a simple route in relatively good to excellent yields in three or four process steps, starting with the dichloride of the formula (II). In the variant according to the invention with three process steps, a phosgenation is avoided.

The compounds of the formula (II) can be prepared by converting a compound of the formula (IX) or a salt thereof 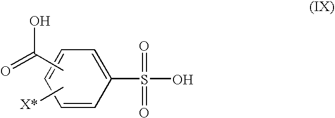
where X* is as defined in formula (II),
with one or more halogenating agents selected from the group consisting of the acid halides of sulfur or phosphorus, in one or more reaction steps, into the compound of the formula (II), preferably the dichloride.

The process defined above is novel and also forms part of the subject matter of the invention. The compounds of the formula (IX) are known, for example, from WO 95/26952, or they can be prepared analogously to the known processes.

Some of the compounds of the formula (II) are known. Thus, U.S. Pat. No. 4,110,373 describes the reaction of unsubstituted or substituted benzotrichlorides with oleum. Mentioned in this publication are, inter alia, the preparations of 4-chloro-3-chlorosulfonylbenzoyl chloride and 3-chloro-5-chlorosulfonylbenzoyl chloride.

Furthermore, NL-A-7603612 discloses the preparation of 2-chlorosulfonylbenzoyl chloride from 2-sulfobenzoic acid by reaction with phosgene as halogenating agent in polar aprotic solvents such as DMF. Here, considerable amounts of dichlorotolylsultone (3,3-dichloro-1,1-dioxobenzo-1-thia-2-oxolane) are formed as byproduct. The process is generally also described for derivatives of sulfobenzoic acids which are additionally halogenated or nitrated on the benzene ring.

The compounds of the formula (II) or the formula (IIa) in which the halosulfonyl group is located in the position ortho to the carbonyl halide group and that in the position para to the carbonyl halide group is additionally halogenated, preferably iodinated, are likewise novel and form part of the subject matter of the invention (compounds (IIa)).

Owing to the side reactions in the known halogenation method with phosgene, it was an object of the present invention to provide an alternative process which allows the preparation of the compounds (II) or (IIa).

Suitable halogenating agents are inorganic acid halides of sulfur and phosphorus, for example thionyl halides, such as thionyl fluoride or thionyl chloride, or sulfuryl halides, such as sulfuryl chloride, or phosphorus halides, such as phosphorus trichloride, phosphoryl chloride, phosphorus pentachloride, phosphorus tribromide. Preference is given to preparing dichlorides (formula (II) or (IIa), where Hal¹=Cl and Hal²=Cl), preferably with thionyl chloride, phosphorus trichloride, phosphoryl chloride or phosphorus pentachloride, in particular with thionyl chloride.

In general, the halogenating agent is employed in an amount of one reaction equivalent per reactive group, or in excess. The customary known reaction equivalents have to be taken into account, for example in the case of the chlorinating agent thionyl chloride 1 reaction equivalent, in the case of PCl₃ and POCl₃ three reaction equivalents and in the case of PCl₅ one or four reaction equivalents, depending on the reaction conditions.

The stoichiometry requires at least 2 equivalents of halogenating agent per mole of the compound of the formula (IX). Depending on the halogenating agent, from 2 to 10 equivalents of halogenating agent per mole of diacid are generally sufficient. In the case of thionyl chloride, preference is given to using from 4 to 8 mol per mole of the compound of the formula (IX). Excess halogenating agent and any hydrogen halide formed are preferably removed from the reaction product or chemically bound, continuously during or after the reaction. In the case of thionyl chloride, it is in most cases possible to remove an excess from the product by distillation.

The halogenation of the compound (IX) is generally carried out in the presence of an inert (relatively) unpolar organic solvent; however, in individual cases it can also be carried out in the absence of a solvent. Suitable solvents are numerous inert solvents, preferably substantially unpolar solvents, which, under the halogenating conditions, do not or do not substantially react with the halogenating agent or the reaction product. Examples of suitable solvents are:
aliphatic and aromatic hydrocarbons, such as, for example, mineral oils, petroleum ether, n-pentane, n-hexane, cyclopentane, cyclohexane or toluene, xylenes, mesitylene, naphthalene derivatives, Solvesso® 200 (high-boiling mixture of aromatic compounds);
halogenated aliphatic and aromatic hydrocarbons, such as methylene chloride, dichloroethane or chlorobenzene, chlorotoluene or dichlorobenzene and mixtures of two or more of the abovementioned solvents or diluents. Suitable inert solvents are advantageously those which can also be used in the preceding step or the subsequent step of the overall process. Suitable for this purpose are, for example, unhalogenated or halogenated aromatic hydrocarbons, preferably high-boiling organic solvents, such as xylene, toluene, mesitylene, chlorobenzene or dichlorobenzene, or mixtures thereof.

The halogenation reaction can be catalyzed by adding polar basic compounds of low nucleophilicity. Suitable for this purpose are, for example, sterically hindered amine bases, for example in the form of trisubstituted amines or nitrogen heterocycles. Suitable in principle are, for example, triethylamine, pyridine, alkylpyridines (for example picolines, lutidines), DBU (1,8-diazabicyclo[5.4.0]undec-7-ene), DABCO (1,4-diazabicyclo[2.2.2]octane) or amides, such as the dialkylformamides DMF (dimethylformamide), diethylformamide, di-n-propylformamide, diisopropylformamide, di-n-butylformamide or di-n-pentylformamide, or dimethylacetamide. For practical reasons such as easier work-up, purification or efficiency, the chosen proportion of catalyst should be as low as possible. Frequently, amounts of from 0.01 to 2 molar equivalents, preferably from 0.02 to 1 molar equivalent, of catalyst, based on the compound of the formula (IX), are sufficient for accelerating the reaction.

Depending on halogenating agent and catalyst, the suitable reaction temperatures may vary and can be determined easily in preliminary experiments. In general, they are in the range of from −10° C. to the boiling point of the solvent in question, preferably from 20° C. to 150° C., in particular from 40° C. to 120° C., the lower limit of the reaction temperature being determined by a detectable conversion.

In principle, the reaction can be carried out by initially charging the starting material of the formula (IX), in most cases neat or dissolved or suspended in the solvent, with the catalyst at an elevated temperature (above the temperature at which a reaction would set in), followed by metered addition of the halogenating agent. Even at elevated temperature, the reaction may be delayed and the reaction may proceed relatively slowly and/or not go to completion.

Alternatively, it is possible to initially charge the halogenating agent, preferably chlorinating agent, with the catalyst at elevated temperature, preferably at from 60 to 80° C., followed by metered addition of the starting material, either as a solid in the absence of a solvent or in suspension in the solvent. This way, it is possible to avoid a large excess of starting material in the reactor.

After the reaction has gone to completion, excess halogenating agent is preferably distilled off, which may, if appropriate, be carried out under reduced pressure. Owing to the reactivity of the dihalide of the formula (II), this is after the preparation preferably directly, without intermediate isolation, processed further, i.e. for example in the case of the monoesterification described above to the compound of the formula (III) or a salt thereof.

In the examples below, the amounts are based on weight unless specifically defined otherwise.

### EXAMPLE 1

### Preparation of a dihalide

### Preparation of 2-chlorosulfonyl-4-iodobenzoyl chloride by addition of solid potassium 3-iodo-6-carboxybenzenesulfonate

Under a blanket of nitrogen, 650 g (5.3 mol) of thionyl chloride (technical grade, >97% pure) are heated to 70° C., and 8 g (0.11 mol) of dimethylformamide are added slowly. After the addition has ended, the mixture is stirred at the stated temperature for 15 minutes, followed by rapid addition, via a screw feeder for solids, of 242 g (0.661 mol) of monopotassium 3-iodo-6-carboxybenzenesulfonate such that a waste gas stream is formed which is easy to control. After the addition has ended, the internal temperature is slowly raised to 85° C. and maintained at this temperature for 2 hours. Excess thionyl chloride is then distilled off at a temperature of 90-100° C. During the distillation, the pressure is reduced down to a minimum of 180 mbar. This gives a melt of the product containing suspended finely crystalline potassium chloride.

To remove remaining thionyl chloride, 100 ml of xylene are added and distilled off under reduced pressure. Following the addition of xylene, the resulting suspension can be used without further work-up for a subsequent esterification. Alternatively, the mixture can be worked up by adding 400 ml of xylene, followed, after cooling to about 30° C., by filtration under protective gas. The precipitate is washed twice with xylene and the combined filtrates are freed from the solvent under reduced pressure. This gives 238 g (0.652 mol=98.6% of theory) of a slightly yellowish solid (purity >99%, HPLC).

### EXAMPLE 2

### Preparation of a dihalide

### Preparation of 2-chlorosulfonyl-4-iodobenzoyl chloride by addition of a suspension of potassium 3-iodo-6-carboxybenzenesulfonate in xylene

Under a blanket of nitrogen, 650 g (5.3 mol) of thionyl chloride (industrial grade, >97% pure) are heated to 70° C., and 8 g (52 mmol) of di-n-butylformamide are added slowly. After the addition has ended, the mixture is stirred at the stated temperature for 15 minutes. The temperature is increased to 85° C., and a thoroughly stirred suspension of finely ground 242 g (0.74 mol) of monopotassium 3-iodo-6-carboxybenzenesulfonate in 400 ml of xylene is then added rapidly such that a waste gas stream is formed which is easy to control. After the addition has ended, the addition funnel is washed with a little xylene and the mixture is kept at the abovementioned temperature for 2 hours. Excess thionyl chloride is then distilled off via a fractionation column at a temperature of 90-100° C. During the distillation, the pressure is reduced down to a minimum of 180 mbar. The distillation is continued until the boiling point of xylene at the still head is stable at the corresponding pressure. If appropriate, xylene removed by distillation is replaced by adding fresh xylene.

The resulting reaction mixture can be worked up as in Example 1 or be used directly for a subsequent esterification.

### EXAMPLE 3

### Preparation of a dihalide

### Preparation of 2-chlorosulfonyl-4-iodobenzoyl chloride by reaction from 4-iodo-2-sulfo-benzoic acid

20.9 g of 4-iodo-2-sulfobenzoic acid are suspended in 80 ml of phosphoryl chloride (POCl₃). 26.7 g of phosphorus pentachloride (PCl₅) are added while stirring. The temperature is raised slowly to 100-110° C., wherein vigorous gas evolution commences as from ca. 40° C. internal temperature. After 1 hour reaction time, the reaction mixture is cooled down to room temperature and the solvent removed at the rotary evaporator. 28.3 g of crude 2-chlorosulfonyl-4-iodobenzoyl chloride are obtained.

### EXAMPLE 4

### Preparation of a compound (III)

### Preparation of methyl 2-chlorosulfonyl-4-iodobenzoate

A reaction mixture obtained as in Example 1 or 2 is cooled to 20-25° C., and 140 ml (110.6 g) of methanol are added dropwise such that the internal temperature does not exceed 28° C. After the addition has ended, the mixture is stirred until the reaction has gone to completion and worked up by variant A or B.

Variant A: Under reduced pressure, excess methanol is removed completely by distillation, together with a little xylene, at a temperature of less than 30° C. The mixture is diluted with xylene to a total volume of 1320 ml and, in three portions, washed with water. The organic phase is separated off and dried by azeotropic distillation under reduced pressure. For a subsequent reaction, the product can be used in the form of the solution in xylene.

Alternatively, the solvent is distilled off under reduced pressure, giving 237 g (=99.5% crude yield*) of the title compound as a solidified melt having a purity of 98% (HPLC), (97.4% of theory*).

*=based on monopotassium 3-iodo-6-carboxybenzenesulfonate employed

Variant B: The mixture is diluted with xylene to a total volume of 1320 ml and, in three portions, washed with water. The organic phase is separated off and dried by azeotropic distillation under reduced pressure. For a subsequent reaction, the product can be used in the form of the solution in xylene.

Alternatively, the solvent is distilled off under reduced pressure, giving 235 g (=98.6% crude yield*) of the title compound as a solidified melt having a purity of 97% (HPLC), (95.6% of theory*).

*=based on monopotassium 3-iodo-6-carboxybenzenesulfonate employed

### EXAMPLE 5

### Preparation of a compound (III) from an acid dichloride

### Preparation of prop-2-inyl 2-chlorosulfonyl-4-iodobenzoate

28.3 g of the crude acid dichloride as obtained by example 4 are dissolved in 150 ml of chloroform and 7.7 ml of propargyl alcohol are added. The mixture is heated for 3 hours at boiling temperature, cooled down to room temperature and poured on to 200 ml of ice water. The aqueous phase is neutralized with NaHCO₃, the phases are separated and the aqueous phase extracted twice more with dichloromethane. The collected organic phases are dried over Na₂SO₄ and the solvent is removed at the rotary evaporator. 23.5 g of crude prop-2-ynyl 2-chlorosulfonyl-4-iodobenzoate are obtained.

### EXAMPLE 6

### Preparation of a compound (III) from a halogenosulfobenzoic acid

### Preparation of prop-2-ynyl 2-chlorosulfonyl-4-iodobenzoate

98.4 g of 4-iodo-2-sulfobenzoic acid are dissolved in a solution of 33.7 g of potassium hydroxide (pellets) in 150 ml of water and 500 ml of methanol. The solvent is then removed at the rotary evaporator, and the residue is dried using high vacuum. 133.9 g of crude di-potasssium salt of the starting material is obtained. 150 ml of thionyl chloride are added dropwise to 66 g of this salt and a further 4.7 ml of dimethylformamide (DMF) are added. The mixture is heated at boiling point until gas evolvement ends, and the solvent is then removed under exclusion of moisture at the rotary evaporator. 150 ml of propargyl alcohol are then added dropwise while cooling, and the mixture is stirred for 6 hours at room temperature. The reaction solution is evaporated, mixed with 150 ml water and filtered off with suction and the filter cake is thoroughly washed with water. After suspending the filter cake in dichloromethane and filtration, the organic phase is concentrated. It contains 35.2 g of prop-2-ynyl 2-chlorosulfonyl-4-iodobenzoate having a melting point of 69-73° C. Additionally, the phase contains 14.3 g 4-iodo-2-sulfobenzoic acid as a hydrolysis product.

### EXAMPLE 7

### Preparation of methyl 4-iodo-[3-(4-methoxy-6-methyl-1,3,5-triazin-2-yl)-ureidosulfonyl]benzoate

106 g of methyl 2-chlorosulfonyl-4-iodobenzoate (97% pure) are dissolved in 500 g of acetonitrile, and 23 g of technical grade sodium cyanate (95%) are added. The mixture is cooled to 6-10° C., and 25 g of pyridine in 100 ml of acetonitrile are added over 24 hours, and the mixture is stirred until the reaction has gone to completion (preparation of the stabilized isocyanate).

43 g of technical grade 2-amino-4-methoxy-6-methyl-1,3,5-triazine are then added, and the mixture is cooled to 0° C. 12 g of dry hydrogen chloride gas are then introduced below the surface, and, after the end of the introduction, the mixture is stirred at 40° C. until the reaction is complete. The acetonitrile is distilled off under reduced pressure and the precipitate is then filtered off and washed with acetonitrile. The precipitate is suspended in a mixture of acetone and dilute hydrochloric acid and filtered once more, washed with water and acetone and dried under reduced pressure. This gives 114 g (77.3% of theory) of the title compound as a white powder (purity: greater than 98%, HPLC).

1. A process for preparing the compound of the formula (I) or a salt thereof 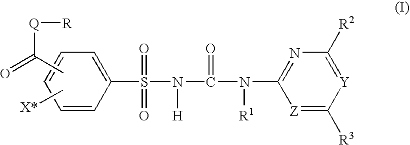
where
Q is oxygen or sulfur,
X* is hydrogen, halogen, cyano, nitro, (C₁-C₃)-alkyl or methoxy,
Y,Z independently of one another are CH or N, where Y and Z are not simultaneously CH,
R is hydrogen, (C₁-C₁₂)-alkyl, (C₂-C₁₀)-alkenyl, (C₂-C₁₀)-alkynyl, (C₁-C₆)-alkyl which is mono- to tetra-substituted by radicals selected from the group consisting of halogen, (C₁-C₄)-alkoxy, (C₁-C₄)-alkylthio, CN, [(C₁-C₄)-alkoxy]carbonyl and (C₂-C₆)-alkenyl, or (C₃-C₈)-cycloalkyl which is unsubstituted or substituted by radicals selected from the group consisting of (C₁-C₄)-alkyl, (C₁-C₄)-alkoxy, (C₁-C₄)-alkylthio and halogen, (C₅-C₈)-cycloalkenyl, phenyl-(C₁-C₄)-alkyl which is unsubstituted in the phenyl radical or substituted by one or more radicals selected from the group consisting of halogen, (C₁-C₄)-alkyl, (C₁-C₄)-alkoxy, (C₁-C₄)-haloalkyl, (C₁-C₄)-alkylthio, [(C₁-C₄)-alkoxy]carbonyl, [(C₁-C₄)-alkyl]carbonyloxy, carbamoyl, [(C₁-C₄)-alkyl]carbonylamino, [(C₁-C₄)-alkyl]aminocarbonyl, di-[(C₁-C₄)-alkyl]aminocarbonyl and nitro, or a radical of the formulae A-1 to A-10 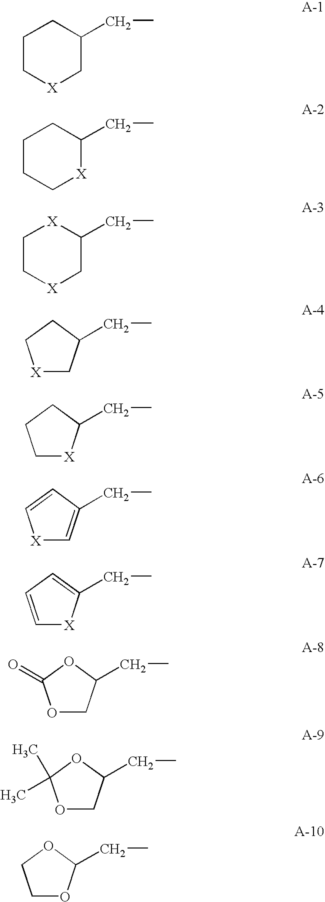
where in the formulae A-1 to A-10
the radical X or the radicals X independently of one another is/are O, S, S(O) or SO₂,
R¹ is hydrogen or (C₁-C₃)-alkyl,
R² is hydrogen, halogen, (C₁-C₃)-alkyl or (C₁-C₃)-alkoxy, where each of the two last-mentioned radicals is unsubstituted or mono- or polysubstituted by halogen or (C₁-C₃)-alkoxy,
R³ is hydrogen, halogen, (C₁-C₃)-alkyl, (C₁-C₃)-alkoxy or (C₁-C₃)-alkylthio, where each of the three last-mentioned radicals is unsubstituted or mono- or polysubstituted by halogen or mono- or disubstituted by (C₁-C₃)-alkoxy or (C₁-C₃)-alkylthio, or a radical of the formula NR⁴R⁵, (C₃-C₆)-cycloalkyl, (C₂-C₄)-alkenyl, (C₂-C₄)-alkynyl, (C₃-C₄)-alkenyloxy or (C₃-C₄)-alkynyloxy,
R⁴ and R⁵ independently of one another are hydrogen, (C₁-C₄)-alkyl, (C₃-C₄)-alkenyl, (C₁-C₄)-haloalkyl or (C₁-C₄)-alkoxy,
which comprises
a) converting a compound of the formula (II) 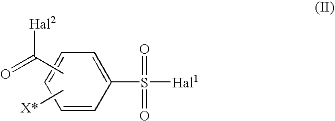
where
Hal¹ is a halogen atom,
Hal² is a halogen atom and
X* is as defined in formula (I)
by reaction with a compound of the formula R-Q-H or a salt thereof into a compound of the formula (III) 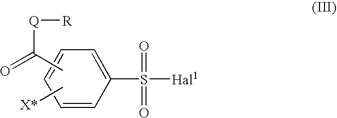
where R, Q and X are as defined in formula (I) and Hal¹ is as defined in formula (II), and
(b) with or without intermediate isolation either
(b1) ammonolysing the resulting compound (III) to give the sulfonamide of the formula (IV) 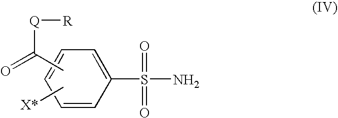
where R, Q and X* are as defined in formula (III), and converting the compound (IV) with or without intermediate isolation with phosgene into the phenylsulfonyl isocyanate of the formula (V) 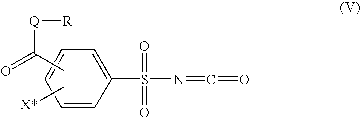
where R, Q and X* are as defined in formula (III), or
(b2) converting the resulting compound (III) with a cyanate into the isocyanate of the formula (V) or a solvated (stabilized) derivative thereof, and
(c) converting the isocyanate of the formula (V) or its stabilized derivative, with or preferably without intermediate isolation, with a heterocyclic amine of the formula (VI) 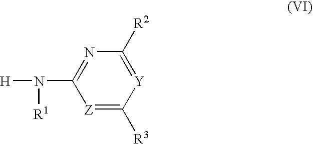
where R¹, R², R³, Y and Z are as defined in formula (I),
into the sulfonylurea of the formula (I) or a salt thereof.

2. The process as claimed in claim 1, wherein in the compound of the formula (I) or its salt
Q is an oxygen atom,
X* is a hydrogen atom or a halogen atom,
R is (C₁-C₄)-alkyl, (C₂-C₄)-alkenyl, (C₂-C₄)-alkynyl, (C₁-C₄)-haloalkyl or (C₁-C₄)-alkoxy(C₁-C₄)-alkyl,
R¹ is a hydrogen atom,
R² is (C₁-C₄)-alkyl or (C₁-C₄)-alkoxy,
R³ is (C₁-C₄)-alkyl or (C₁-C₄)-alkoxy,
Y is a nitrogen atom or a group of the formula CH and
Z is a nitrogen atom.

3. The process as claimed in claim 1, wherein in step a) the compounds of the formula (II) used are compounds of the formula (IIa): 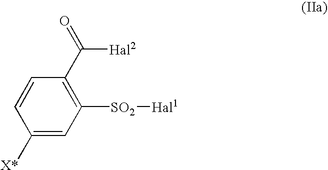
where
Hal¹ is halogen,
Hal² is halogen, and
X* is as defined in formula (I).

4. The process as claimed in claim 1, wherein in the compound of the formula (I) or its salt
X* is an iodine atom,
R is methyl or ethyl,
R² is methoxy,
R³ is methyl and
Y is a nitrogen atom.

5. A process as claimed in claim 1, wherein the esterification to the monoester (III) is carried out in an inert organic solvent selected from the group of the nonpolar aprotic organic solvents, at a temperature of from −20° C. to 100° C.

6. The process as claimed in claim 1, wherein the esterification to the monoester (III) is carried out using a (C₁-C₄)-alkanol at a temperature of from −10° C. to 70° C. or using an alkali metal (C₁-C₄)-alkoxide at a temperature of from −20° C. to 50° C.

7. The process as claimed in claim 1, wherein the preparation of the isocyanate (V) is carried out in the presence of an aprotic polar solvent at a temperature of from −30° C. to 70° C.

8. The process as claimed in claim 7, wherein the preparation of the isocyanate (V) is carried out in the presence of an N-heteroaromatic compound.

9. A compound of the formula (VIII) 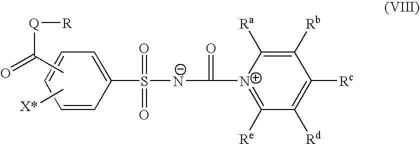
where R, Q and X* are as defined in formula (I) as set forth in claim 1 and R^{a}, R^{b}, R^{c}, R^{d} and R^{e} are each independently of one another hydrogen, (C₁-C₆)-alkyl, (C₂-C₆)-alkenyl, (C₂-C₆)-alkynyl or (C₁-C₆)-alkoxy or two adjacent radicals together with the linking carbon atoms of the first ring form a fused-on carbocyclic ring having 4 to 8 carbon atoms or a heterocyclic ring having 4 to 8 ring atoms and 1, 2 or 3 heteroring atoms selected from the group consisting of N, O and S.

10. A process for preparing compounds of the formula (VIII) as defined in claim 9, which comprises reacting a compound of the formula (III) 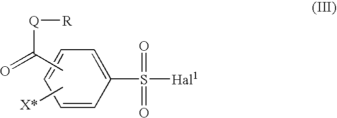
where Hal¹ is a halogen atom
with a cyanate in the presence of a compound of the formula (VII) 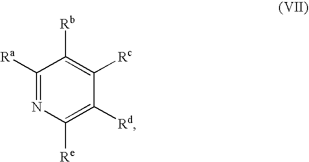
where in the formulae (III) and (VII) the radicals R, R^{a}, R^{b}, R^{c}, R^{d}, R^{e}, Q and X* are as defined in formula (VIII).

11. A process for preparing compounds of the formula (II) 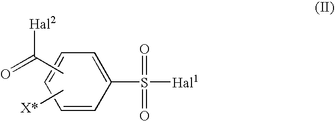
where Hal¹ and Hal² are each independently of one another a halogen atom and X* is hydrogen, halogen, cyano, nitro, (C₁-C₃)-alkyl or methoxy, which comprises converting a compound of the formula (IX) or a salt thereof 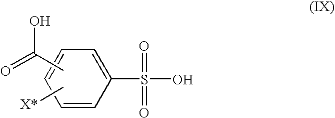
where X* is as defined in formula (II)
with one or more halogenating agents selected from the group of the inorganic acid halides of sulfur or phosphorus, in one or more reaction steps, into the compound of the formula (II).

12. The process as claimed in claim 11, wherein
the halogenating agent used is thionyl fluoride, thionyl chloride, sulfuryl chloride, phosphorus trichloride, phosphoryl chloride, phosphorus pentachloride or phosphorus tribromide.

13. The process as claimed in claim 11, wherein the process is carried out in the presence of an inert organic solvent and a catalyst selected from the group of the sterically hindered amine bases.

14. The process as claimed in claim 11, wherein the reaction temperature is in the range from 20° C. to 150° C.

15. A compound of the formula (IIa) 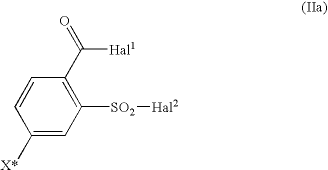
where Hal¹, Hal² and X* are each independently of one another a halogen atom and X* is an iodine atom.

16. The compound as claimed in claim 15, wherein Hal¹ and Hal² are each a chlorine atom and X* is an iodine atom.

17. A process for preparing a compound of the formula (I), or a salt thereof, as defined in claim 1, wherein a compound of the formula (III) 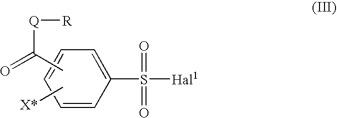
where R and Q are as defined in formula (I) and X* is as defined in formula (I), is converted with a cyanate into the isocyanate of the formula (V) 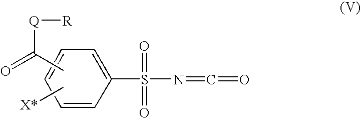
where R, Q and X* are as defined in formula (III),
or a solvated (stabilized) derivative thereof, and
the resulting compound (V) or its stabilized derivative is converted with a heterocyclic amine of the formula (VI) 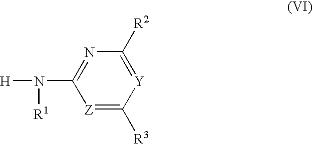
where R¹, R², R³, Y and Z are as defined in formula (I),
into the sulfonylurea of the formula (I) or a salt thereof.
